(19) United States
(12) Patent Application Publication
(10) Pub. No.: US 2026/0263210 A1
(43) Pub. Date: Sep. 10, 2026

(54) IMPLANTABLE CONTAINER FOR AUGMENTATION PROCEDURES

(71) Applicant: OTS Medical Ltd., Tel-Aviv (IL)

(72) Inventors: Assaf DEKEL, Savyon (IL); Adely LEVY, Pardes Hanna-Karkur (IL); Ruth ICEKSON, Tel Aviv (IL); Eyal SANDACH, Yahud (IL)

(73) Assignee: OTS Medical Ltd., Tel-Aviv (IL)

(21) Appl. No.: 19/073,071

(22) Filed: Mar. 7, 2025

(51) Int. Cl. A61F 2/08; A61L 27/16; A61L 27/18; A61L 27/24; A61L 27/36; A61L 27/38; A61L 27/54; A61L 27/56; A61L 27/58

(52) U.S. Cl. CPC A61F 2/0805; A61L 27/16; A61L 27/18; A61L 27/24; A61L 27/3616; A61L 27/3834; A61L 27/54; A61L 27/56; A61L 27/58; A61F 2002/0888; A61F 2250/0003; A61L 2300/604; A61L 2430/10

(57) ABSTRACT

The invention discloses an implantable device for augmenting and/or reinforcing a tissue that has been fixed, comprising a body, optionally a fillable body, comprising an internal area; and optionally at least one organic material within said internal area of said body; where the fillable body comprises one or more mechanical characteristics similar to said tissue that has been fixed into which said implantable device is implanted.

### FIELD AND BACKGROUND OF THE INVENTION

The present invention, in some embodiments thereof, relates to a soft-tissue augmentation device and related methods thereof; and, more particularly, but not exclusively, to a soft-tissue augmentation device configured for repairing damaged tissue and related methods thereof.

Additional background art includes U.S. Pat. No. 9,248,012 disclosing a fluid delivery system is used for the treatment of subcutaneous hernia sites and includes a bioabsorbable polymer fabric mesh layer and a bioabsorbable and permeable bladder attached to the upper side of the fabric mesh layer. Fluid from the bladder is diffused through pores communicating between the bladder and the fabric mesh layer so as to be dispersed to underlying tissue.

U.S. Pat. No. 8,178,745 disclosing a medical plaster which is able to supply damaged skin and open wounds with an active compound which helps to accelerate wound healing and allows it to proceed better. The plaster has novel design features which enable, in particular, protein- or peptide-containing medicaments, such as, for example, erythropoietin (EPO), to exert their wound-healing or wound-healing-promoting action by release of the active compound from the plaster without being significantly deactivated or metabolized in the process by substances occurring in the wound secretion.

U.S. Patent Application No. US20130218104A1 disclosing systems, apparatuses, and methods that employ a medical device to augment delivery of an agent to biological tissue. The medical device includes a reservoir containing the agent, a release port, and sealable base that can be attached to the biological tissue so that the agent is held under pressure upon attachment. The device can only release the agent through the release port. A penetrating instrument can be utilized to augment delivery of the agent to the biological tissue.

U.S. Patent Application No. US20130267971A1 disclosing a novel single plane tissue repair device such as a patch. The device has a base member with an opening therethrough, and a closure member associated with the opening. The mesh has a bioabsorbable polymeric adhesion barrier attached to the bottom side of the base member about its periphery to form a pocket that is accessible through the opening. The mesh may be used in open surgical procedures for hernia repairs and other repairs of body wall defects.

### SUMMARY OF THE INVENTION

According to an aspect of some embodiments of the present invention there is provided an implantable device for augmenting and/or reinforcing a tissue that has been fixed, comprising:
a. a fillable body comprising an internal area; and
b. at least one organic material within said internal area of said body;
wherein said fillable body comprises one or more mechanical characteristics similar to said tissue that has been fixed into which said implantable device is implanted.

According to some embodiments of the invention, said fillable body comprises walls having pores.

According to some embodiments of the invention, said pores comprise a size of from 50 μm to 300 μm.

According to some embodiments of the invention, said pores are sized and shaped to allow said at least one organic material to migrate out of said fillable body.

According to some embodiments of the invention, said pores are sized and shaped to allow said at least one organic material to create interactions with said tissue.

According to some embodiments of the invention, said fillable body is made of one or more of Polylactic Acid (PLA), Polyglycolic Acid (PGA), Poly (lactic-co-glycolic acid) (PLGA), Polycaprolactone (PCL), Collagen, Collagen-based materials, hydrogels, Polyethylene Terephthalate (PET), Polyester; Polypropylene (PP), Expanded Polytetrafluoroethylene (ePTEE) and Ultra High Molecular Weight Polyethylene (UHMWPE).

According to some embodiments of the invention, one mechanical characteristic of said one or more mechanical characteristics is Elastic Modulus (Young's Modulus); and wherein said Elastic Modulus is from 500 to 1500 megapascals (MPa).

According to some embodiments of the invention, one mechanical characteristic of said one or more mechanical characteristics is Strain; and wherein said Strain is from 5% to 20%.

According to some embodiments of the invention, one mechanical characteristic of said one or more mechanical characteristics is Viscoelasticity; and wherein said body is made of a Viscoelastic material.

According to some embodiments of the invention, said body comprises a length of from 20 mm to 40 mm.

According to some embodiments of the invention, said body comprises a width of from 3 mm to 35 mm.

According to some embodiments of the invention, further comprising at least one means for attaching said implantable device to said tissue.

According to some embodiments of the invention, said at least one means is one or more of sutures, organic glues and bone anchors.

According to some embodiments of the invention, said body further comprises at least one coating.

According to some embodiments of the invention, said plurality of organic materials are one or more of bone marrow, bone marrow stem cells, osteogenic cells, pre-osteoblasts, osteoblasts, mesenchymal stem cells, bone marrow mesenchymal stem cells, blood derivatives like Platelet-rich plasma (PRP) and/or plasma-derived medicinal products (PDMPs) and/or Platelet-rich fibrin (PRF) and pluripotent stem cells.

According to some embodiments of the invention, said plurality of organic materials are one or more of autografts, allografts and synthetic grafts.

According to some embodiments of the invention, said tissue is a tendon.

According to some embodiments of the invention, further comprising one or more bone anchors attached to said fillable body.

According to some embodiments of the invention, at least one part of said fillable body is configured to be used as a soft anchor.

According to an aspect of some embodiments of the present invention there is provided a method for augmenting and/or reinforcing a tissue that has been fixed, comprising:
a. filling an implantable device with at least one organic material; and
b. implanting said filled implantable device on said tissue.

According to some embodiments of the invention, further comprising, before said filling, identifying a type of implantable device required.

According to some embodiments of the invention, said identifying comprises identifying a type of implantable device made of a material that comprises mechanical characteristics that are similar to mechanical characteristics of said tissue.

According to some embodiments of the invention, further comprising, before said implanting, fixing said tissue.

According to some embodiments of the invention, said implanting comprises suturing said implantable device to said tissue.

According to some embodiments of the invention, said implanting further comprises:
a. implanting a bone anchor attached to a first side of said implantable device into a bone;
b. extending said implantable device over said tissue; and
c. suturing a second side of said implantable device in said tissue.

According to some embodiments of the invention, said implanting further comprises:
a. implanting a first bone anchor attached to a first side of said implantable device into a first bone;
b. extending said implantable device over said tissue; and
c. implanting a second bone anchor attached to a second side of said implantable device into a second bone.

According to some embodiments of the invention, said first bone and said second bone are a same bone.

According to some embodiments of the invention, said implanting comprises:
a. implanting a part of said implantable device as a soft anchor into a bone;
b. extending said implantable device over said tissue; and
c. suturing a second side of said implantable device in said tissue.

According to some embodiments of the invention, said implanting further comprises:
a. implanting a first part of said implantable device as a soft anchor into a first bone;
b. extending said implantable device over said tissue; and
c. implanting a second part of said implantable device as a soft anchor into a second bone.

According to some embodiments of the invention, said first bone and said second bone are a same bone.

According to an aspect of some embodiments of the present invention there is provided an implantable device for reinforcing a tissue that has been fixed, comprising a body comprising one or more mechanical characteristics similar to said tissue that has been fixed into which said implantable device is implanted.

According to some embodiments of the invention, said body comprises walls having pores.

According to some embodiments of the invention, said pores comprise a size of from 50 μm to 300 μm.

According to some embodiments of the invention, said fillable body is made of one or more of Polylactic Acid (PLA), Polyglycolic Acid (PGA), Poly (lactic-co-glycolic acid) (PLGA), Polycaprolactone (PCL), Collagen, Collagen-based materials, hydrogels, Polyethylene Terephthalate (PET), Polyester; Polypropylene (PP), Expanded Polytetrafluoroethylene (ePTEE) and Ultra High Molecular Weight Polyethylene (UHMWPE).

According to some embodiments of the invention, one mechanical characteristic of said one or more mechanical characteristics is Elastic Modulus (Young's Modulus); and wherein said Elastic Modulus is from 500 to 1500 megapascals (MPa).

According to some embodiments of the invention, one mechanical characteristic of said one or more mechanical characteristics is Strain; and wherein said Strain is from 5% to 20%.

According to some embodiments of the invention, one mechanical characteristic of said one or more mechanical characteristics is Viscoelasticity; and wherein said body is made of a Viscoelastic material.

According to some embodiments of the invention, said body comprises a length of from 20 mm to 40 mm.

According to some embodiments of the invention, said body comprises a width of from 3 mm to 35 mm.

According to some embodiments of the invention, further comprising at least one means for attaching said implantable device to said tissue.

According to some embodiments of the invention, said at least one means is one or more of sutures, organic glues and bone anchors.

According to some embodiments of the invention, said body further comprises at least one coating.

According to some embodiments of the invention, said tissue is a tendon.

According to some embodiments of the invention, further comprising one or more bone anchors attached to said body.

According to some embodiments of the invention, at least one part of said body is configured to be used as a soft anchor.

According to an aspect of some embodiments of the present invention there is provided a method for reinforcing a tissue that has been fixed, comprising implanting said implantable device on said tissue.

According to some embodiments of the invention, further comprising identifying a type of implantable device required.

According to some embodiments of the invention, said identifying comprises identifying a type of implantable device made of a material that comprises mechanical characteristics that are similar to mechanical characteristics of said tissue.

According to some embodiments of the invention, further comprising, before said implanting, fixing said tissue.

According to some embodiments of the invention, said implanting comprises suturing said implantable device to said tissue.

According to some embodiments of the invention, said implanting further comprises:
a. implanting a bone anchor attached to a first side of said implantable device into a bone;
b. extending said implantable device over said tissue; and
c. suturing a second side of said implantable device in said tissue.

According to some embodiments of the invention, said implanting further comprises:
a. implanting a first bone anchor attached to a first side of said implantable device into a first bone;
b. extending said implantable device over said tissue; and
c. implanting a second bone anchor attached to a second side of said implantable device into a second bone.

According to some embodiments of the invention, said first bone and said second bone are a same bone.

According to some embodiments of the invention, said implanting comprises:
a. implanting a part of said implantable device as a soft anchor into a bone;
b. extending said implantable device over said tissue; and
c. suturing a second side of said implantable device in said tissue.

According to some embodiments of the invention, said implanting further comprises:
a. implanting a first part of said implantable device as a soft anchor into a first bone;
b. extending said implantable device over said tissue; and
c. implanting a second part of said implantable device as a soft anchor into a second bone.

According to some embodiments of the invention, said first bone and said second bone are a same bone.

According to an aspect of some embodiments of the present invention there is provided a method of treatment of a soft tissue comprising using and/or implanting a device as described herein, by one or more of the methods described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings/images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1a is a schematic representation of an exemplary implantable device, according to some embodiments of the invention;

FIG. 1b is a schematic representation of an exemplary implantable device implanted on a tissue requiring augmentation and/or repair, according to some embodiments of the invention;

FIGS. 2a-b are an image of an exemplary body of a device and a zoom-in of exemplary pores sizes, according to some embodiments of the invention;

FIGS. 3a-b are schematical representations of the mechanical response of an exemplary implantable device to actions of the tissue, according to some embodiments of the invention;

FIGS. 4a-b are schematic representations of exemplary anchoring methods, according to some embodiments of the invention;

FIGS. 5a-b are schematic representations of exemplary additional anchoring methods, according to some embodiments of the invention;

FIGS. 6a-g are schematic representations of exemplary devices without the insertion of organic materials therein, according to some embodiments of the invention;

FIG. 7 is a flowchart of an exemplary method of use of an exemplary device, according to some embodiments of the invention, and

FIG. 8 is a flowchart of an exemplary method of use of an exemplary device without filling it with organic materials, according to some embodiments of the invention.

### DESCRIPTION OF SPECIFIC EMBODIMENTS OF THE INVENTION

The present invention, in some embodiments thereof, relates to a soft-tissue augmentation device and related methods thereof; and, more particularly, but not exclusively, to a soft-tissue augmentation device configured for repairing damaged tissue and related methods thereof.

### Overview

An aspect of some embodiments of the invention relates to an implantable device configured to for augmenting and/or reinforce tissues, for example soft-tissues, while providing the necessary organic materials/tissue for repairing the tissue where the implantable device is positioned. In some embodiments, organic materials can be, for example, one or more of bone marrow, bone marrow stem cells, osteogenic cells, pre-osteoblasts, osteoblasts, mesenchymal stem cells, bone marrow mesenchymal stem cells, blood derivatives like Platelet-rich plasma (PRP) and/or plasma-derived medicinal products (PDMPs) and/or Platelet-rich fibrin (PRF) and pluripotent stem cells (which in general will be referred hereinafter just as “organic materials”). In some embodiments, the implantable device comprises a body comprising (housing and/or enclosing) one or more types of cells and/or organic materials. In some embodiments, the body comprises a sack-like configuration having porous walls. In some embodiments, the implantable device is configured to be attached to the tissue (for example, soft-tissue), for example by means of sutures, organic glues or any other attaching means. In some embodiments, the implantable device is held in position by the natural space within the body and/or by tissues located in the area where the device is implanted. In some embodiments, the device comprises at least one anchor (optionally two anchors) configured to be attached to a bone. In some embodiments, the device is configured to be attached on one side to a bone, then the body is extended over the soft tissue, and the other side of the sac is either attached to the soft tissue itself, or to another bone using a second anchor. In some embodiments, the body is made of a hollowed mesh or a hollowed polyester woven having porous having a minimum size, for example from about 100 μm to about 150 μm, that allows the organic material to migrate or create interactions with the tissues (for example, soft tissues), where the implantable device is implanted. In some embodiments, the size of the porous is chosen to be sufficient to facilitate the migration of the organic materials which will then promote vascularization and tissue integration. In some embodiments, sizes of the porous can vary depending on various factors, for example, the specific application and the type of organic material (cells or tissues) involved. For example, larger pores might be necessary in some contexts to support more robust tissue ingrowth and vascularization, while smaller pores might be sufficient in other contexts where finer cellular interactions are required.

In some embodiments, the body of the implantable device is absorbable and/or biodegradable. In some embodiments, the minimum absorbable and/or biodegradable time for the materials used in the body of the device can vary depending on the specific application and material properties. For example, the materials can be one or more of: Polylactic Acid (PLA), which degrades over a period of 6 months to 2 years. However, modifications in its formulation can be done to alter the degradation rate; Polyglycolic Acid (PGA), which usually degrades faster than PLA, often within 4 to 6 months and it is commonly used in sutures and other fast-resorbing applications; Poly (lactic-co-glycolic acid) (PLGA), which, by adjusting the ratio of lactic acid to glycolic acid, allows to change the degradation time from a few months to over a year. PLGA with a higher glycolic acid content tends to degrade faster; Polycaprolactone (PCL, which is known for its slower degradation rate, often taking 1 to 2 years to fully degrade, and it is optionally used in applications where long-term support is required; Collagen and/or Collagen-based materials that are configured to degrade over a few weeks to several months, depending on their crosslinking and formulation; and Hydrogels, in which the degradation time can vary widely based on their composition, crosslinking density, and environmental conditions, that allow to have a device that can degrade in a range from a few days to several months. In some embodiments, the materials used in the implantable devices are configured to provide structural support for a minimum of a few weeks to a few months before beginning to degrade. In some embodiments, the specific timeframe will depend on the required healing period and the nature of the tissue being augmented and/or repaired.

In some embodiments, the implantable device is suitable for the augmentation and/or repair of soft-tissues like tendons. Without being bound to theory, the elasticity of tendons is typically characterized by their ability to stretch and return to their original length. This property is quantified by the elastic modulus, also known as the Young's modulus. Tendons are highly elastic and resilient tissues, primarily composed of collagen fibers, which give them their unique mechanical properties. The key characteristics of tendons are one or more of: 1. Elastic Modulus (Young's Modulus): the elastic modulus of tendons generally ranges from 500 to 1500 megapascals (MPa). This variability depends on the specific tendon, its location in the body, and the health and age of the individual. 2. Strain: tendons can typically withstand a strain (deformation relative to their original length) of up to 8-10% before experiencing damage. Beyond this range, tendons may undergo micro-damage, leading to a higher risk of injury. 3. Viscoelastic Properties: tendons exhibit viscoelastic behavior, meaning they have both elastic and time-dependent (viscous) responses to stress. This property allows tendons to absorb and dissipate energy, which is crucial during activities involving rapid or repetitive movements. These properties have functional implications that need to be taken into consideration when assessing the type of device and/or type of organic materials to be used. For example: A. Energy Storage and Return: Tendons store elastic energy during muscle contraction and release it during muscle relaxation, enhancing the efficiency of movement. B. Shock Absorption: The viscoelastic properties help in shock absorption, protecting muscles and bones from sudden impacts. C. Force Transmission: Tendons efficiently transmit forces from muscles to bones, enabling movement and stability. In some embodiments, the body of the implantable device is designed to match and/or mimic the physical requirements of the tissue onto which the device is implanted. For example, in the case of soft tissues like tendons, which are characterized by extending and shrinking, the body of the implantable device is configured to extend and shrink together with the tendon. In some embodiments, the incorporation time for grafts in tendons and ligaments can vary depending on several factors, including the type of graft, the surgical technique used, the specific tendon or ligament being repaired, and the individual patient's healing response. Generally, the timeline for graft incorporation in tendons and ligaments includes several key phases:

1. Inflammatory Phase (1-2 weeks): Initial response to surgery includes inflammation, which helps remove damaged tissue and initiate healing. Blood flow to the area increases, and immune cells begin to clear out debris.

2. Proliferative Phase (2-6 weeks): Cells begin to proliferate and produce new collagen fibers. Vascularization of the graft increases, and the initial scaffold of the new tissue is formed. During this phase, the graft is still relatively weak and prone to damage.

3 Early Remodeling Phase (6-12 weeks): Collagen fibers begin to organize and align with the stress lines of the tendon or ligament. The graft gradually integrates with the surrounding tissue. Strength of the graft starts to improve, but it remains vulnerable to excessive stress.

4. Late Remodeling Phase (3-6 months): Continued maturation and strengthening of the collagen fibers. The structure of the graft becomes more similar to the native tendon or ligament. Gradual return to normal activities, but heavy or high-impact activities are still limited.

5. Maturation Phase (6-12 months and beyond): Final maturation and consolidation of the graft. The graft achieves near-normal strength and functionality. Full return to athletic activities or heavy labor might be possible, depending on individual progress and the specific tendon or ligament repaired.

In some embodiments, an exemplary tendon graft incorporation process, such as those used in ligament reconstruction surgeries (e.g., ACL reconstruction), can be described as follows:

1 Early Healing and Integration: Duration: 4 to 6 weeks, the graft undergoes initial incorporation, with revascularization and cellular repopulation beginning.

2. Intermediate Remodeling: Duration: 3 to 6 months, the graft continues to remodel, with collagen fibers reorganizing and adapting to the mechanical environment.

3. Mature Integration: Duration: 6 months to 1 year or longer, full integration and remodeling are achieved, with the graft attaining mechanical properties similar to the original tendon or ligament.

In some embodiments, an exemplary tendons and ligaments incorporation process, such as those used in anterior cruciate ligament (ACL) reconstruction), can be described according to the type of grafts, as follows:

1. Autografts: These grafts, harvested from the patient's own body (e.g., hamstring or patellar tendon), typically show faster incorporation due to better initial biocompatibility. Initial healing is observed within 6-8 weeks, with full maturation taking up to 6-12 months.

2. Allografts: Grafts from a donor (cadaveric tissue) may take longer to incorporate due to potential immune response and slower revascularization. Initial healing can take 8-12 weeks, and full incorporation can extend to 12-18 months.

3. Synthetic Grafts: These vary widely in incorporation times. Early-generation synthetic grafts often faced issues with laxity and stability, but newer materials show improved results. Generally, synthetic grafts take longer to fully integrate compared to biological options, sometimes requiring additional support from surrounding tissues.

In some embodiments, the implantable device is generated according to the specific needs, taking under consideration the abovementioned incorporation processes, type of graft and type of tissue in need to be augmented and/or repaired.

In some embodiments, the device, or at least parts thereof, are suitable to be used as soft anchors and be attached to a bone without the need for further attaching means. In some embodiments, in addition of being used as a soft anchor, the device comprises integral attaching means, for example, strings or sutures.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In some embodiments, the exemplary implantable device is configured to be a single use device intended for reinforcement and/or repair of soft tissues that are repaired by suture or other fixation devices during soft tissue repair surgery including, for example, reinforcement of rotator cuff, patellar, Achilles, biceps, quadriceps tendon or other tendons. In some embodiments, the implantable device is optionally not intended to replace normal body structure or provide the full mechanical strength to support the rotator cuff, patellar, Achilles, biceps, quadriceps tendon or other tendons.

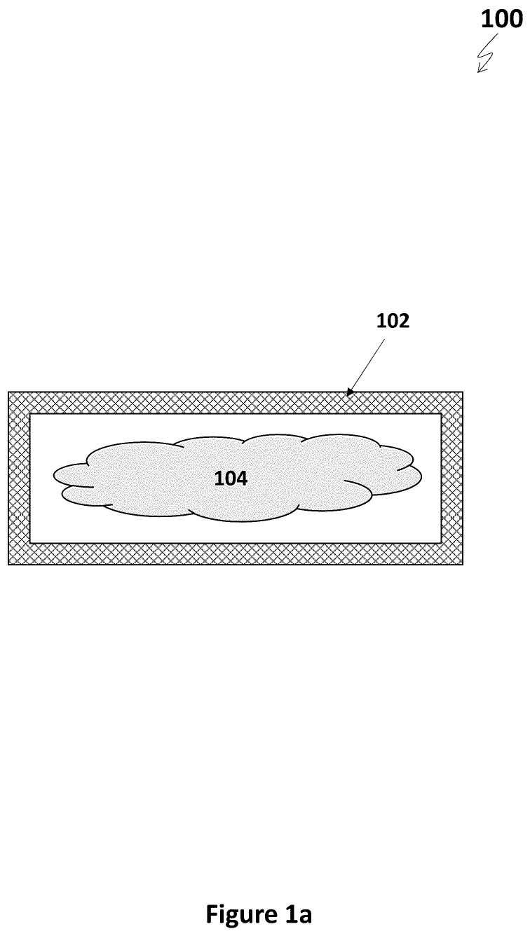

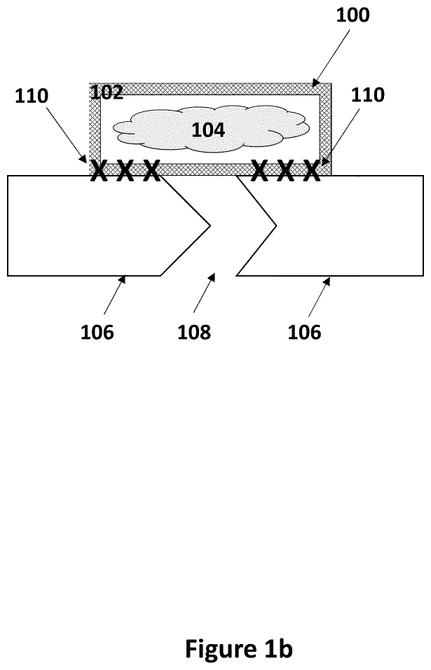

Referring now to FIG. 1a, showing a schematic representation of an exemplary implantable device, according to some embodiments of the invention. In some embodiments, an exemplary implantable device 100 comprises a hollowed body 102 which is filled with at least one organic material 104. In some embodiments, for example, the implantable device 100 is a woven Polyester implant that has a tubular structure of flat width of 5.7±0.3 mm, for example from about 5 mm to about 35 mm, a double wall thickness 0.44±0.1-mm and a length of 25±1 mm or 30±1 mm or 35±1 mm or 45±1 mm or 58±1 mm. In some embodiments, the device is woven with 26±5 picks per inch. For example, the device comprises a width of from about 3 mm to about 45 mm, a double wall thickness of from about 0.2 mm to about 0.45 mm, a length of from about 20 mm to about 130 mm, and picks per inch from about 20 to about 40. In some embodiments, the device is characterized by an elasticity of about 3%-20%. Tensile strength of about 600N-900N.

In some embodiments, the device is sterilized by use of Gamma radiation or ETO.

Referring now to FIG. 1b, showing a schematic representation of an exemplary implantable device implanted on a tissue requiring augmentation and/or repair, according to some embodiments of the invention. In some embodiments, the implantable device 100 is configured to be implanted within a body of a patient in order to augment and/or repair a tissue 106. In some embodiments, the tissue is a soft-tissue, like a tendon. In FIG. 1b, the tissue 106 is shown with a schematic tear 108 in need of repair. In some embodiments, the implantable device 100 is anchored to the tissue 106 by means of sutures 110. In some embodiments, the sutures can be: 1. an intrinsic part of the implantable device 100, 2. external sutures which do not form part of the implantable device 100, or 3. a combination thereof. In some embodiments, the implantable device 100 is anchored to the tissue 106 by means other than sutures, for example, organic glues, or others as will be further disclosed below.

### Exemplary Fixation Methods

1. General Fixation Principles: In some embodiments, the device is secured using one or more fixation techniques tailored to different anatomical locations, surgical approaches, and biomechanical needs. In some embodiments, the fixation methods ensure one or more of: Strong mechanical stability to enhance tendon repair; Minimally invasive applicability for arthroscopic, percutaneous, and open surgery; Promotion of biological integration through bioactive and resorbable fixation options; and Load-sharing mechanics to distribute forces evenly across the repair site.

2. Universal Fixation Methods: In some embodiments, one or more of the following fixation methods can be applied across various soft tissues (for example tendons), either individually or in combination, depending on the clinical scenario:

### Suture-Based Fixation

Direct Tendon Suturing (Onlay or Wrap-Around): the device is sutured to the native tendon using side-to-side sutures (simple, mattress, or locking-loop) and/or circumferential suturing for complete wrapping around the tendon. In some embodiments, the device is sutures using high-strength non-absorbable sutures or bioresorbable sutures.

### Tendon-to-Bone Fixation (Footprint Augmentation)

In some embodiments, the device is sutured directly to the tendon and anchored to the bone attachment site. In some embodiments, the surgery can be performed arthroscopically, percutaneously, or through open surgery. In some embodiments, a double-row suture technique may be used to distribute load and enhance integration.

### Anchor-Based Fixation

Suture Anchors: In some embodiments, suture anchors are used. In some embodiments, the device is secured via tendon-to-bone suture anchors at its lateral edges. In some embodiments, the device is secured via medial attachment to tendon remnant, lateral attachment to bone footprint, the device is secured using biooabsorbable or metal anchors may be used for enhanced fixation.

Knotless Anchor Fixation: In some embodiments, a device is preloaded into a knotless anchor system for simplified fixation. In some embodiments, tension can be adjusted dynamically to optimize tendon-bone healing.

### Adhesive-Assisted Fixation

In some embodiments, biologic adhesives (fibrin glue, platelet-rich fibrin, collagen-based adhesives) can be used as a supplementary or primary fixation method. In some embodiments, biologic adhesives can be applied in minimally invasive repairs or adjunctively with sutures/anchors. In some embodiments, using biologic adhesives potentially reduces risk of stress concentrations from sutures and promotes natural tendon healing.

### Press-Fit or Interference Fixation

Tendon-Bone Interference Screws: In some embodiments, the device can be compressed into a bone tunnel using an interference screw. In some embodiments, common in ligamentous and tendon reconstructions (e.g., ACL, patellar tendon).

Compression Button or Loop Fixation: In some embodiments, the device can be secured between cortical bone fixation points using suture buttons. In some embodiments, this allows adjustable tensioning and dynamic tissue adaptation.

### Percutaneous and Endoscopic Fixation

In some embodiments, minimally invasive suturing or stapling methods can be employed for smaller tendons or delicate structures. In some embodiments, endoscopic-assisted deployment of anchors or sutures can be used in deep tendon repairs (e.g., flexor/extensor tendons).

Exemplary application Across Various Tendons

In some embodiments, the fixation of the device can be applied to multiple tendons, with specific optimizations for each anatomical site, for example:

Rotator Cuff (RC) Repair: Primary Repair Augmentation: Patch secured onlay with sutures or anchors. Bridging Repair for Irreparable Tears: Patch sutured between tendon and tuberosity. Superior Capsule Reconstruction (SCR): Patch secured between glenoid and humerus for humeral head stabilization.

Achilles Tendon Repair: Primary Tendon Repair Reinforcement: Patch wrapped and sutured circumferentially. Tendon-to-Bone Fixation: Patch anchored to calcaneal footprint using suture anchors. Percutaneous Augmentation: Minimal incision technique with sutures or adhesives.

Patellar and Quadriceps Tendon Repair: Patellar Tendon Augmentation: Patch secured via suture anchors in the patella. Quadriceps Tendon Augmentation: Patch sutured directly into bone tunnels.

Flexor and Extensor Tendon Augmentation (Hand and Wrist): Circumferential Suturing: Patch is wrapped and secured with fine sutures. Microsurgical Approaches: Endoscopic delivery of sutures for seamless integration.

Biceps Tendon Repair: Biceps Tenodesis: Patch reinforces the tendon within the bicipital groove via suture anchors. Biceps Tenotomy Augmentation: Patch provides structural support when reattachment is not performed.

In some embodiments, the implantable device 100 is naturally anchored in place by the tissues surrounding the area in need of augmentation and/or repair and/or by a natural space in the location of implantation.

### Exemplary Organic Materials

In some embodiments, exemplary organic materials used for the augmentation and/or repair of tissues are one or more of bone marrow, bone marrow stem cells, osteogenic cells, pre-osteoblasts, osteoblasts, mesenchymal stem cells, bone marrow mesenchymal stem cells, blood derivatives like Platelet-rich plasma (PRP) and/or plasma-derived medicinal products (PDMPs) and/or Platelet-rich fibrin (PRF) and pluripotent stem cells.

In some embodiments, the organic materials can be harvested from the patient's own body (called: Autografts), can be harvested from a donor (called allografts), can be synthetic grafts, or a combination thereof.

### Exemplary Characteristics of the Body 102 of the Implantable Device

In some embodiments, the characteristics of the body 102 of the implantable device are changed and/or chosen depending on the required needs of the device.

### Exemplary Pore Size

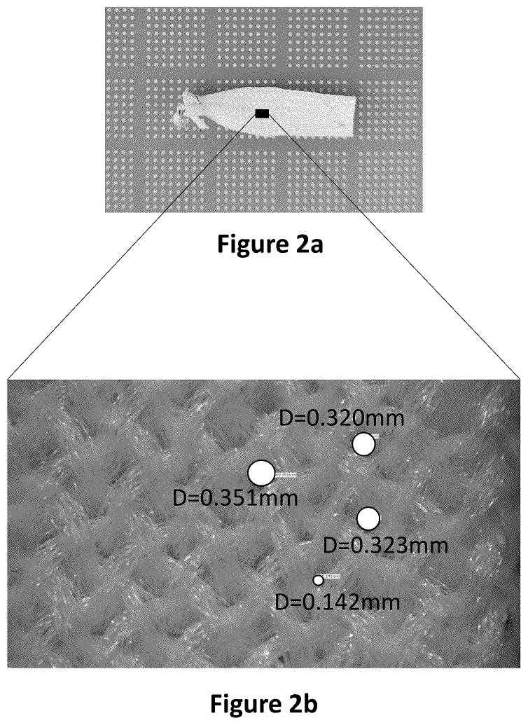

Referring now to FIGS. 2a-2b, showing an image of an exemplary body of a device and exemplary pores sizes, according to some embodiments of the invention. In general, an exemplary body is made of a material that is woven polyester and/or mesh, as shown for example in the image in FIG. 2a, having pores that allow for the organic materials to migrate and/or create interactions with soft tissues, as shown for example in the image in FIG. 2b. In some embodiments, the pores in the mesh are from about 100 μm to about 150 μm, optionally from about 80 μm to about 200 μm, optionally from about 50 μm to about 300 μm. In some embodiments, the size of the pores. In some embodiments, the size of pores is chosen according to the specific application and/or the type of cells and/or tissues involved. For example, larger pores might be necessary in some contexts to support more robust tissue ingrowth and vascularization, while smaller pores might be sufficient in other contexts where finer cellular interactions are required.

### Exemplary Materials

In some embodiments, the materials chosen for the body 102 of the device are according to the minimum and/or required absorbable and/or biodegradable time of the materials and depending on the specific application and other material properties.

Exemplary, non-limiting, guidelines for common biodegradable materials are as follows:
1. Polylactic Acid (PLA): Typically, PLA degrades over a period of 6 months to 2 years. However, modifications in its formulation can alter its degradation rate.
2. Polyglycolic Acid (PGA): PGA usually degrades faster than PLA, often within 4 to 6 months. It's commonly used in sutures and other fast-resorbing applications.
3. Poly (lactic-co-glycolic acid) (PLGA): By adjusting the ratio of lactic acid to glycolic acid, the degradation time of PLGA can range from a few months to over a year. PLGA with a higher glycolic acid content tends to degrade faster.
4. Polycaprolactone (PCL): PCL is known for its slower degradation rate, often taking 1 to 2 years to fully degrade. It's used in applications where long-term support is required.
5. Collagen: Collagen-based materials can degrade over a few weeks to several months, depending on their crosslinking and formulation.
6. Hydrogels: The degradation time of hydrogels can vary widely based on their composition, crosslinking density, and environmental conditions. They can degrade in a range from a few days to several months.

In some embodiments, the materials used are designed to provide structural support for a minimum of a few weeks to a few months before beginning to degrade. In some 5 embodiments, the specific timeframe will depend on the required healing period and the nature of the tissue being augmented.

| Device | Advantages | Disadvantages | Materials |
|---|---|---|---|
| Synthetic | High strength and | May increase risk | Polyethylene |
| Augmentation | durability | of infection or | Terephthalate (PET) - |
| Devices | Often inert, reducing | foreign body | Polyester |
| | risk of adverse | reaction | Polypropylene (PP) |
| | reactions | Potential for long- | Expanded |
| | Can provide stable | term complications | Polytetrafluoroethylene |
| | support and | such as erosion or | (ePTEE) |
| | reinforcement to | migration | Ultra High Molecular |
| | tissues | May require | Weight Polyethylene |
| | Immediate and | surgical removal in | (UHMWPE) |
| | durable mechanical | some cases | |
| | support. | | |
| Biodegradable | Naturally degrade | Variable | Polylactic Acid (PLA) |
| Augmentation | over time, reducing | degradation rates | Polyglycolic Acid (PGA) |
| Devices | risk of long-term | may not match tissue | Polycaprolactone (PCL |
| | complications | healing | Polydioxanone (PDO) |
| | May promote | Mechanical | |
| | tissue ingrowth and | strength may | |
| | remodeling | decrease as material | |
| | Eliminate need for | degrades | |
| | surgical removal | Risk of | |
| | Provide temporary | inflammatory | |
| | support and are | response during | |
| | gradually absorbed. | degradation process | |
| | Pediatric applications | | |
| | where growth is a | | |
| | factor | | |
| Absorbable | Eliminate need for | Mechanical | |
| Augmentation | surgical removal | properties may not | |
| Devices | Absorbable | be suitable for long- | |
| | materials can reduce | term support | |
| | risk of infection and | Variable | |
| | foreign body reaction | absorption rates may | |
| | Can provide | not match tissue | |
| | temporary support | healing | |
| | during tissue healing | Risk of premature | |
| | | degradation or loss | |
| | | of strength | |
| Bioactive | Promote tissue | Complex | Biologic Scaffolds |
| Augmentation | integration and | manufacturing | Collagen-Based Devices |
| Devices | regeneration | processes may | Decellularized Dermal |
| | Enhance | increase cost | Allografts |
| | biocompatibility and | Effectiveness may | Bio inductive Implants |
| | reduce risk of adverse | vary depending on | Porcine-Derived Tissue |
| | reactions | patient factors and | Matrix |
| | Can stimulate | application | |
| | specific cellular | Long-term effects | |
| | responses for improved | and biocompatibility | |
| | healing | need further research | |
| | | Enhance natural | |
| | | healing processes | |
| | | and tissue | |
| | | regeneration. | |
| Engineering | Tailored design to | Complex | |
| Constructs | match specific | manufacturing | |
| | anatomical | processes and | |
| | requirements | customization may | |
| | Can incorporate | increase cost | |
| | multiple materials for | Requires thorough | |
| | optimized mechanical | understanding of | |
| | and biological | biomechanics and | |
| | properties | tissue engineering | |
| | Allow for precise | principles | |
| | control over scaffold | Long-term | |
| | architecture and | performance and | |
| | porosity | integration need | |
| | | extensive testing | |
| Composite | Combine properties | Complex | |
| Devices | of multiple materials | interactions between | |
| | for enhanced | different materials | |
| | performance | may introduce | |
| | Can address | unforeseen | |
| | multiple clinical needs | complications | |
| | simultaneously | Manufacturing | |
| | Offer versatility in | and regulatory | |
| | design and application | approval may be | |
| | | more challenging | |
| | | due to diverse | |
| | | components | |
| | | Long-term | |
| | | performance and | |
| | | biocompatibility | |
| | | need comprehensive | |
| | | evaluation | |

### Exemplary Mechanical Characteristics of the Body of the Device

In some embodiments, the mechanical characteristics of the body of the device are chosen and/or designed to match the mechanical characteristics of the tissue where the implantable is going to be implanted.

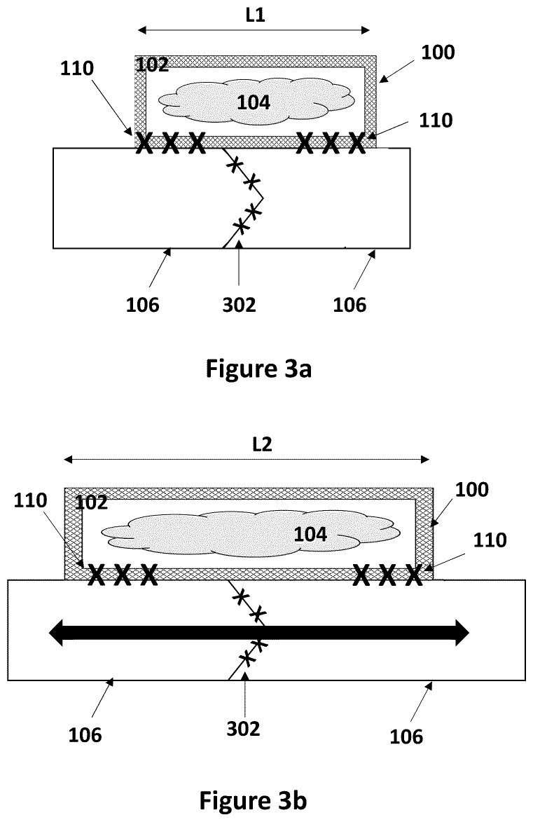

Referring now to FIGS. 3a-b showing schematical representations of the mechanical response of an exemplary implantable device to actions of the tissue, according to some embodiments of the invention.

In some embodiments, as mentioned above, the implantable device is configured to be positioned in a soft-tissue for augmentation and/or repair, after the soft tissue has been repaired, for example by suturing. In FIG. 3a, a soft tissue 106 has been repaired using sutures 302. The implantable device 100 has been implanted into the soft-tissue 106 by attaching the body 102 to the tissue 106 using sutures 110.

In some embodiments, the body 102 of the implantable device 100 is configured to extend and shrink (move in general) together with the tissue 106. For example, in the case of soft tissues, like tendons, the elasticity of tendons is typically characterized by their ability to stretch and return to their original length. This property is quantified by the elastic modulus, also known as the Young's modulus. Tendons are highly elastic and resilient tissues, primarily composed of collagen fibers, which give them their unique mechanical properties. FIG. 3a shows the tendon 106 at rest, having a first length L1. FIG. 3b shows the tendon 106 extended, having a second length L2, and the implantable device 100, adapting itself to the new length.

Exemplary Key Characteristics of the tissues which are also common to the implantable device are one or more of:

Elastic Modulus (Young's Modulus): The elastic modulus of tendons generally ranges from 500 to 1500 megapascals (MPa). This variability depends on the specific tendon, its location in the body, and the health and age of the individual.

Strain: Tendons can typically withstand a strain (deformation relative to their original length), which is calculated for example:

$$Strain ( ε ) = \frac{Δ L}{{L}_{0}} ,$$

where ΔL is the change in length and L₀ is the original length of the tissue, of up to 8-20% before experiencing damage. Beyond this range, tendons may undergo micro-damage, leading to a higher risk of injury. In some embodiments, the device is designed to withstand a strain of from about 5% to about 20%.

Viscoelastic Properties: Tendons exhibit viscoelastic behavior, meaning they have both elastic and time-dependent (viscous) responses to stress. This property allows tendons to absorb and dissipate energy, which is crucial during activities involving rapid or repetitive movements. In some embodiments, the body 102 is made of viscoelastic materials.

### Exemplary Functional Implications:

Energy Storage and Return: Tendons store elastic energy during muscle contraction and release it during muscle relaxation, enhancing the efficiency of movement.

Shock Absorption: The viscoelastic properties help in shock absorption, protecting muscles and bones from sudden impacts.

Force Transmission: Tendons efficiently transmit forces from muscles to bones, enabling movement and stability.

### Exemplary Incorporation Time for Grafts in Soft Tissues

In some embodiments, the incorporation time for grafts in tendons and ligaments can vary depending on several factors, including the type of graft, the surgical technique used, the specific tendon or ligament being repaired, and the individual patient's healing response. Generally, and without being bound to theory, the timeline for graft incorporation in tendons and ligaments includes several key phases:

Inflammatory Phase (1-2 weeks): Initial response to surgery includes inflammation, which helps remove damaged tissue and initiate healing. Blood flow to the area increases, and immune cells begin to clear out debris.

Proliferative Phase (2-6 weeks): Cells begin to proliferate and produce new collagen fibers. Vascularization of the graft increases, and the initial scaffold of the new tissue is formed. During this phase, the graft is still relatively weak and prone to damage.

Early Remodeling Phase (6-12 weeks): Collagen fibers begin to organize and align with the stress lines of the tendon or ligament. The graft gradually integrates with the surrounding tissue. Strength of the graft starts to improve, but it remains vulnerable to excessive stress.

Late Remodeling Phase (3-6 months): Continued maturation and strengthening of the collagen fibers. The structure of the graft becomes more similar to the native tendon or ligament. Gradual return to normal activities, but heavy or high-impact activities are still limited.

Maturation Phase (6-12 months and beyond): Final maturation and consolidation of the graft. The graft achieves near-normal strength and functionality. Full return to athletic activities or heavy labor might be possible, depending on individual progress and the specific tendon or ligament repaired.

### Exemplary Tendon Graft Incorporation

In the case of tendon grafts, such as those used in ligament reconstruction surgeries (e.g., ACL reconstruction), the integration process can be described as follows:

Early Healing and Integration: Duration: 4 to 6 weeks; The graft undergoes initial incorporation, with revascularization and cellular repopulation beginning.

Intermediate Remodeling: Duration: 3 to 6 months; The graft continues to remodel, with collagen fibers reorganizing and adapting to the mechanical environment.

Mature Integration: Duration: 6 months to 1 year or longer; Full integration and remodeling are achieved, with the graft attaining mechanical properties similar to the original tendon or ligament.

### Exemplary Incorporation Time Based on Type of Graft

In some embodiments, the incorporation time for grafts in soft tissues, like tendons and ligaments, such as those used in anterior cruciate ligament (ACL) reconstruction, varies depending on the type of graft. For example:
1. Autografts: These grafts, harvested from the patient's own body (e.g., hamstring or patellar tendon), typically show faster incorporation due to better initial biocompatibility. Initial healing is observed within 6-8 weeks, with full maturation taking up to 6-12 months.
2. Allografts: Grafts from a donor (cadaveric tissue) may take longer to incorporate due to potential immune response and slower revascularization. Initial healing can take 8-12 weeks, and full incorporation can extend to 12-18 months.
3. Synthetic Grafts: These vary widely in incorporation times. Early-generation synthetic grafts often faced issues with laxity and stability, but newer materials show improved results. Generally, synthetic grafts take longer to fully integrate compared to biological options, sometimes requiring additional support from surrounding tissues.

### Exemplary Characteristics and Applications of Different Embodiments of the Implantable Device

In some embodiments, the implantable device is defined by the type of device. In some embodiments, in selecting the appropriate type of device, surgeons consider the specific clinical scenario, desired outcomes, patient factors, and material properties to ensure optimal healing and functionality. For example, implantable devices can be one or more of: synthetic devices; biodegradable devices; absorbable devices; bioactive devices; engineering constructs and composite devices.

### Exemplary Synthetic Devices

In some embodiments, exemplary synthetic devices are made of one or more of Polyethylene Terephthalate (PET)-Polyester; Polypropylene (PP); Expanded Polytetrafluoroethylene (ePTEE) and Ultra High Molecular Weight Polyethylene (UHMWPE). In some embodiments, potential advantages of using synthetic devices is that they provide high strength and durability, they are often inert, thereby reducing the risk of adverse reactions, they can potentially provide stable support and reinforcement to tissues and/or they provide immediate and durable mechanical support. In some embodiments, potential disadvantages of synthetic devices are that they may increase the risk of infection or cause a foreign body reaction, they may cause long-term complications such as erosion or migration; they may require surgical removal.

Exemplary indications for use of synthetic devices:

| | Indications | Key | | |
|---|---|---|---|---|
| Device | for Use | Features | Pros | Cons |
| Poly-Tape | Rotator cuff, | Extremely strong, | Immediate | Risk of foreign |
| (Xiros) | Achilles | resistant to wear | mechanical | body reaction, |
| | tendon, | | strength, | infection |
| | ACL/PCL | | long-term | |
| | repairs | | stability, | |
| | | | well-tolerated | |
| | | | by the body | |
| Infinity- | ACL/PCL, | High tensile | Immediate | Risk of foreign |
| Lock (Xiros) | rotator cuff, | strength, | mechanical | body reaction, |
| | patellar, | biocompatible, | strength, | infection |
| | Achilles | long-term | long-term | |
| | tendon repairs | reinforcement | stability, | |
| | | | well-tolerated | |
| | | | by the body | |
| Leeds-Keio | ACL | High tensile | Immediate | Risk of foreign |
| Ligament | reconstruction, | strength, | mechanical | body reaction, |
| (Xiros) | rotator cuff | durability, | strength, | infection |
| | repair, Achilles | biocompatibility | long-term | |
| | tendon repair, | | stability, | |
| | patellar tendon | | well-tolerated | |
| | repair | | by the body | |
| LARS | ACL, PCL, | Highly durable, | Immediate | Potential for |
| Ligament | lateral | flexible, strong, | mechanical | foreign body |
| | ligament | biocompatible | support, | reaction, |
| | reconstruction, | | longevity, | stiffness |
| | rotator cuff | | minimal | |
| | repair, Achilles | | stretch | |
| | tendon repair | | | |
| Gore-Tex | Soft tissue | Biocompatibility, | Low tissue | Expensive, |
| Soft Tissue | repair, hernia | minimal tissue | adhesion, | potential for |
| Patch | repair, tendon | reaction, strong | strong, | infection, less |
| | reinforcement, | | minimal | flexibility |
| | ligament | | inflammation | |
| | reinforcement | | | |
| FiberWire/ | Tendon repairs | Extremely strong, | High tensile | High cost, |
| FiberTape | (e.g., rotator | resistant to wear, | strength, | potential for |
| | cuff, Achilles), | minimal | minimal | cutting through |
| | ligament | elongation | elongation, | soft tissue if |
| | repairs (e.g., | | durability | not used |
| | ACL, PCL) | | | properly |

### Exemplary Biodegradable Devices

In some embodiments, exemplary biodegradable devices are made of one or more of Polylactic Acid (PLA), Polyglycolic Acid (PGA), Polycaprolactone (PCL) and Polydioxanone (PDO). In some embodiments, potential advantages of using biodegradable devices is that they naturally degrade over time, reducing risk of long-term complications, they may promote tissue ingrowth and remodeling, they potentially eliminate the need for surgical removal, they potentially provide temporary support and are gradually absorbed and they are potentially useful in pediatric applications where growth is a factor. In some embodiments, potential disadvantages of using biodegradable devices are variable degradation rates which may not match tissue healing, the mechanical strength may decrease as material degrades and there might be a risk of inflammatory responses during degradation process.

### Exemplary Absorbable Devices

In some embodiments, the device is made of bioabsorbable materials. In some embodiments, potential advantages of using absorbable devices are one or more of potentially eliminating the need for surgical removal, the absorbable materials can reduce risk of infection and foreign body reaction and they can provide temporary support during tissue healing. In some embodiments, potential disadvantages of using an absorbable device are that mechanical properties may not be suitable for long-term support, variable absorption rates may not match tissue healing and there might be a risk of premature degradation or loss of strength.

### Exemplary Bioactive Devices

In some embodiments, exemplary bioactive devices are made of one or more of biologic scaffolds, collagen-based devices, decellularized dermal allografts, bioinductive implants and porcine-derived tissue matrix. In some embodiments, potential advantages of using bioactive devices are the potential promotion of tissue integration and regeneration, the potential enhancement in biocompatibility and reduction of risk of adverse reactions and it can potentially stimulate specific cellular responses for improved healing. In some embodiments, potential disadvantages of using bioactive devices are that they have a complex manufacturing process that may increase cost, the effectiveness may vary depending on patient factors and application, long-term effects and biocompatibility need further research, and it may enhance natural healing processes and tissue regeneration.

### Exemplary Engineering Constructs

In some embodiments, potential advantages of using engineering constructs are one or more of: they are tailored design to match specific anatomical requirements, they can incorporate multiple materials for optimized mechanical and biological properties and they potentially allow for precise control over scaffold architecture and porosity. In some embodiments, potential disadvantages of using engineering constructs are that they have a complex manufacturing processes and customization may increase cost, they might require thorough understanding of biomechanics and tissue engineering principles and the long-term performance and integration need extensive testing.

### Exemplary Composite Devices

In some embodiments, potential advantages of using composite devices are one or more of: they potentially combine properties of multiple materials for enhanced performance, they can potentially address multiple clinical needs simultaneously and they can potentially offer versatility in design and application. In some embodiments, potential disadvantages of using composite devices are that complex interactions between different materials may introduce unforeseen complications, manufacturing and regulatory approval may be more challenging due to diverse components and long-term performance and biocompatibility need comprehensive evaluation.

### Exemplary Coatings

In some embodiments, the body 102 is coated with one or more materials that contribute to the scope of the device. For example, the body 102 is coated with one or more of: organic adhesion materials, pharmaceuticals, antibacterial, antifouling, coatings (Prevent Infection & Biofilm Formation), bioactive coatings (promote tissue integration) and drug-eluting coatings (Controlled Drug Release).

### Exemplary Anchoring Methods

In some embodiments, as mentioned above, the device can be anchored directly to the soft tissue, as shown for example in FIG. 1b.

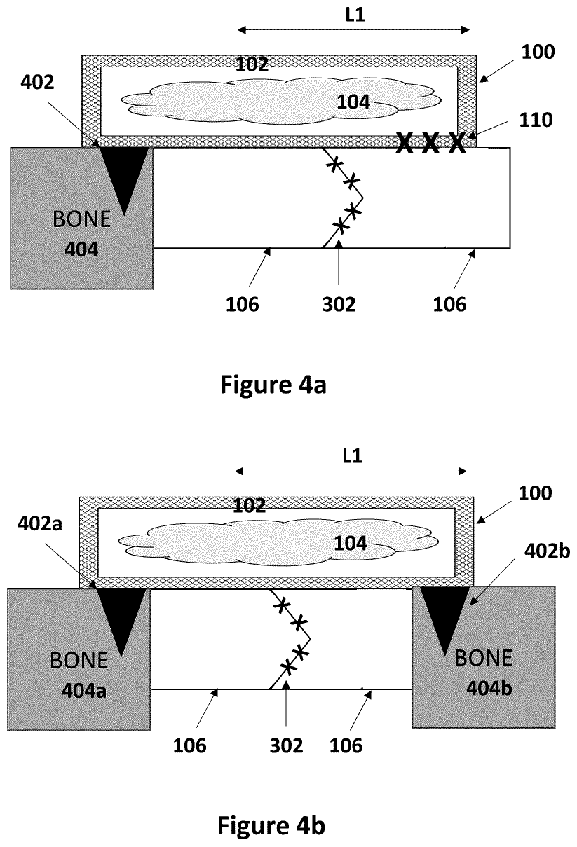

In some embodiments, the device comprises one bone anchor attached to one side of the device, as shown for example in FIG. 4a. In some embodiments, a bone anchor 402 attached to one side of the device is anchored to a bone 404. In some embodiments, the body 102 of the device 100 is extended over the tissue 106 and the other side of the body 102 of the device 100 is sutured 110 over the soft tissue 106.

In some embodiments, the device comprises two (or more) bone anchors attached to each side of the device, as shown for example in FIG. 4b. In some embodiments, a first bone anchor 402a attached to one side of the device is anchored to a first bone 404a. In some embodiments, the body 102 of the device 100 is extended over the tissue 106. In some embodiments, a second bone anchor 402b attached to the other side of the device is anchored to a second bone 404b.

In some embodiments, parts of the body 102 of the device 100 are designed to function as soft anchor.

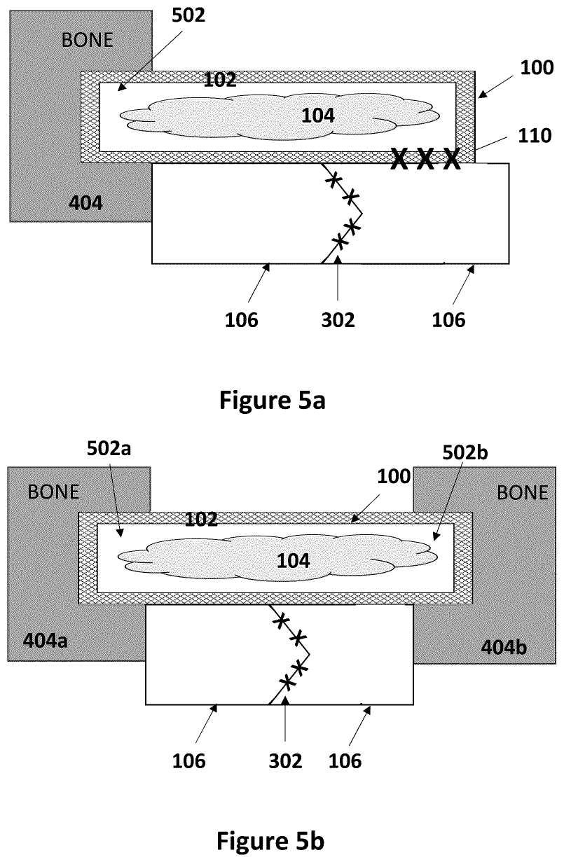

In some embodiments, the device itself (or at least a part of the device) functions as a soft anchor 502 configured to be inserted into a bone and anchor the device to the bone 404, as shown for example in FIG. 5a. In some embodiments, the rest of the body 102 of the device 100 (which does not function as a soft anchor) is extended over the tissue 106 and the other side of the body 102 of the device 100 is sutured 110 over the soft tissue 106.

In some embodiments, two parts of the device (for example at two opposed sides of the body 102) function as soft anchors 502a/502b, as shown for example in FIG. 4b. In some embodiments, a first soft anchor 502a is anchored to a first bone 404a. In some embodiments, the body 102 of the device 100 is extended over the tissue 106. In some embodiments, a second soft anchor 502b is anchored to a second bone 404b.

Exemplary Device without Organic Materials

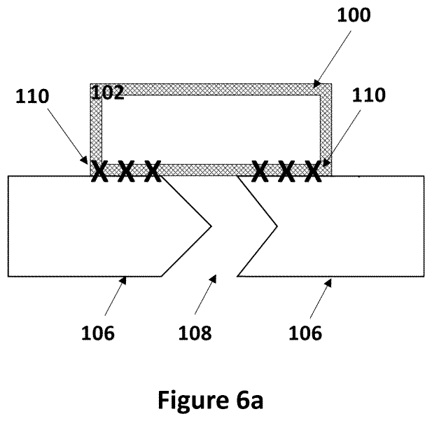

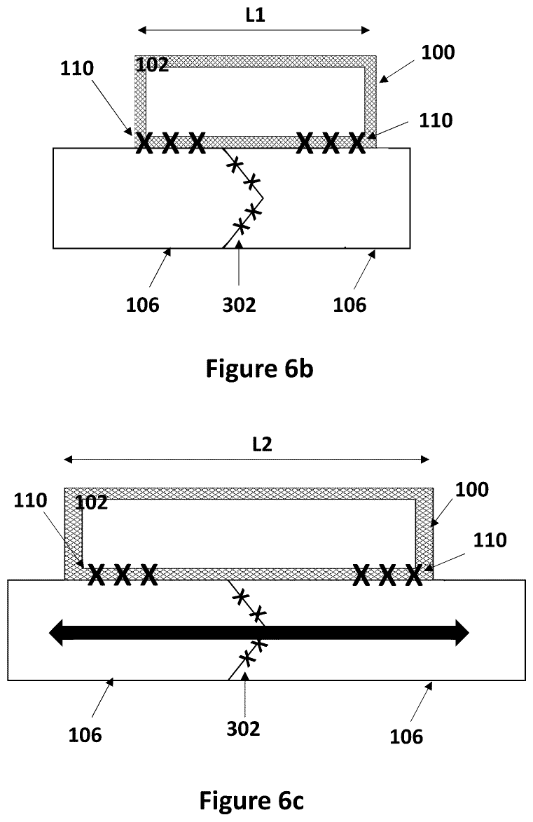

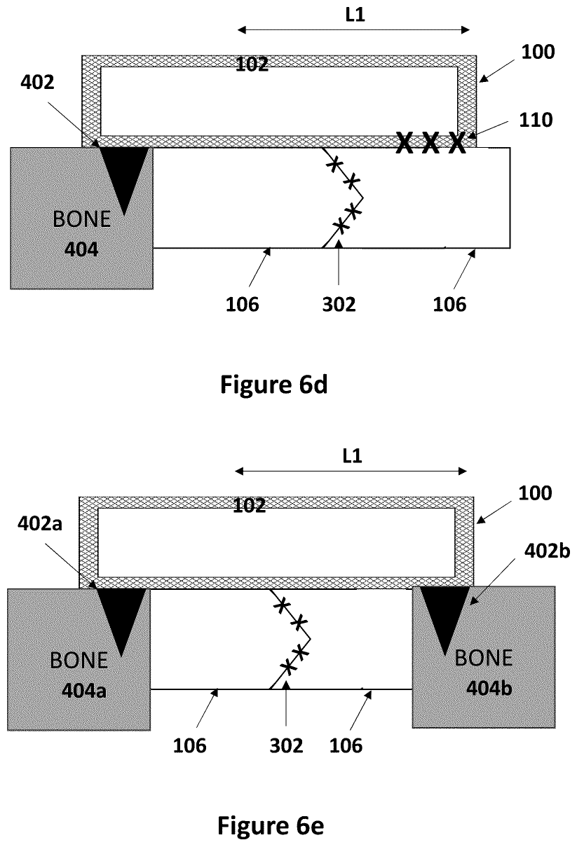

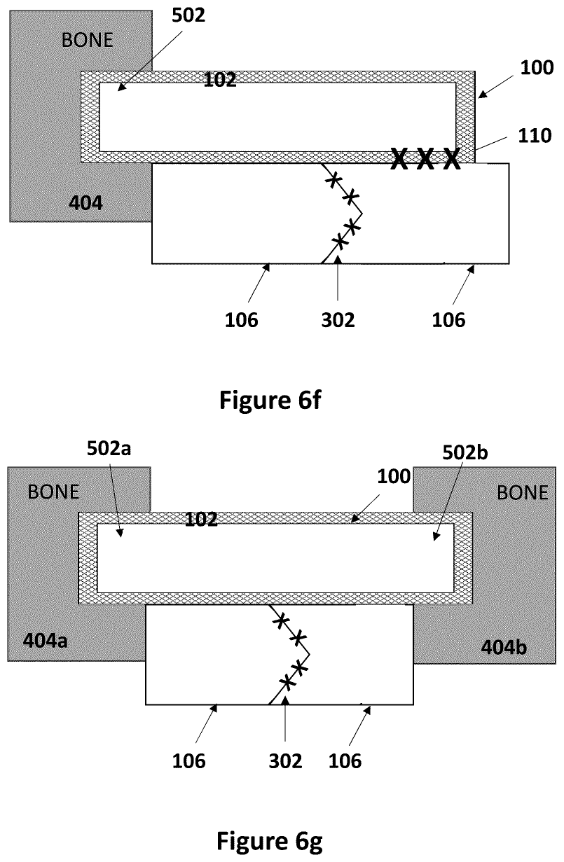

In some embodiments, the device 100 as described herein is implanted without the insertion of organic materials within the body 102 of the device 100. In some embodiments, in these cases, the device acts as reinforcement to the repair on the soft tissue. FIGS. 6a-g show the same embodiments as above but without the organic materials inserted within the body 102.

### Exemplary Methods

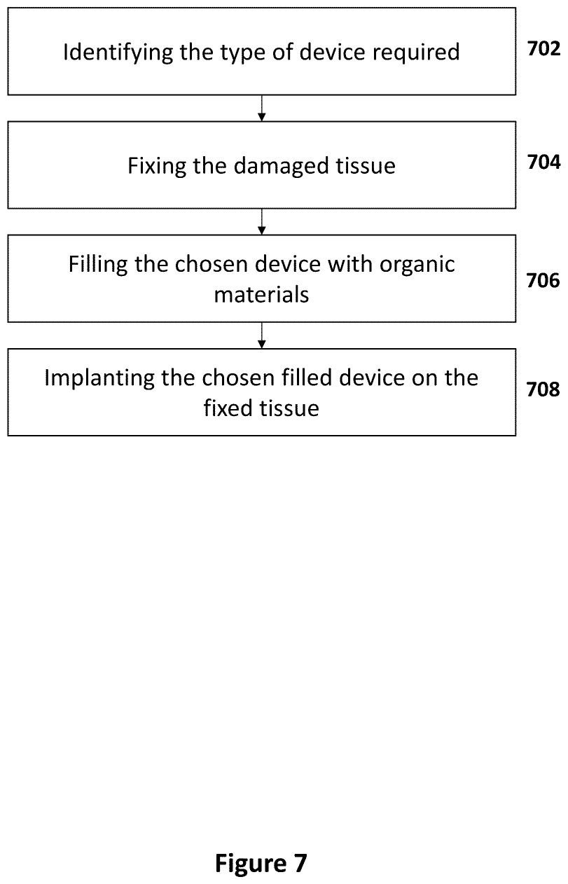

Referring now to FIG. 7, showing a flowchart of an exemplary method of using an exemplary device, according to some embodiments of the invention.

In some embodiments, in general, an exemplary method of treating a fixed tissue comprises implanting a filled device on a fixed tissue, where the device comprises mechanical characteristics similar to that of the fixed tissue. In some embodiments, an exemplary method of use of an exemplary device, comprises one or more of the following actions:
1. Identifying the type of device required (702).
2. Fixing the damaged tissue (704).
3. Filling the chosen device with organic materials (706).
4. Implanting the chosen filled device on the fixed tissue (708).

In some embodiments, the implanting comprises suturing the device on the tissue. In some embodiments, the implanting comprises anchoring a first side of the device to a bone and suturing the device on the tissue. In some embodiments, the implanting comprises anchoring a first anchor to a first bone, extending the device over the tissue and anchoring a second anchor into a second bone. In some embodiments, the first bone and the second bone are the same bone. In some embodiments, the anchoring comprises anchoring a part of a body of the device as a soft anchor into the bone. In some embodiments, two parts of the body can be used as soft anchors and two parts of the body are attached to bones.

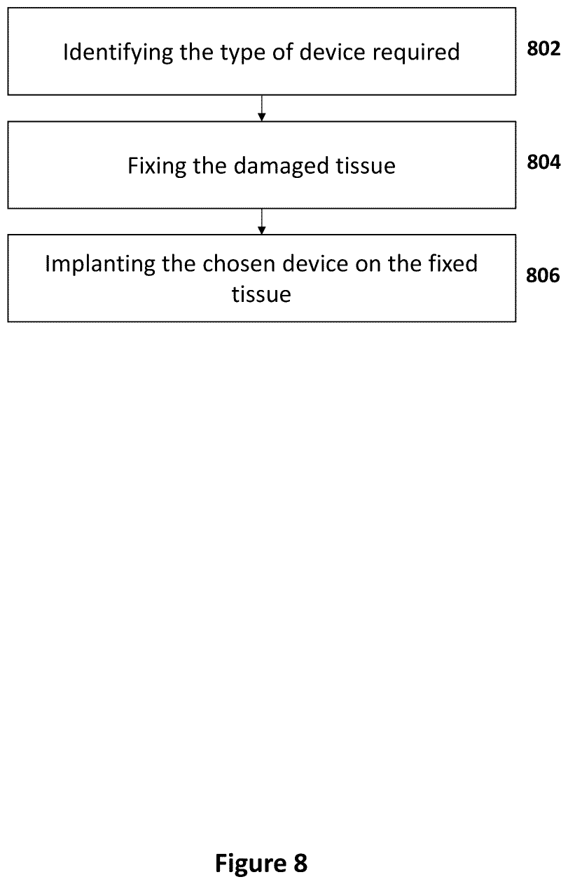

Referring now to FIG. 8, showing a flowchart of an exemplary method of using an exemplary device, according to some embodiments of the invention.

In some embodiments, in general, an exemplary method of treating a fixed tissue comprises implanting a device without filing it with organic material on a fixed tissue, where the device comprises mechanical characteristics similar to that of the fixed tissue. In some embodiments, an exemplary method of use of an exemplary device, comprises one or more of the following actions:
1. Identifying the type of device required (802).
2. Fixing the damaged tissue (804).
4. Implanting the chosen device on the fixed tissue (806).

In some embodiments, the implanting comprises suturing the device on the tissue. In some embodiments, the implanting comprises anchoring a first side of the device to a bone and suturing the device on the tissue. In some embodiments, the implanting comprises anchoring a first anchor to a first bone, extending the device over the tissue and anchoring a second anchor into a second bone. In some embodiments, the first bone and the second bone are the same bone. In some embodiments, the anchoring comprises anchoring a part of a body of the device as a soft anchor into the bone. In some embodiments, two parts of the body can be used as soft anchors and two parts of the body are attached to bones.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An implantable device for augmenting and/or reinforcing a tendon and/or a ligament inside a body of patient that has been fixed, comprising:
a. a fillable body configured to be implanted on a tendon and/or a ligament inside said body and comprising an internal area; and
b. at least one organic material within said internal area of said body; and
wherein said fillable body comprises walls having pores comprising a size of from 50 μm to 300 μm;
wherein said pores are sized and shaped to allow said at least one organic material to migrate out of said fillable body; and
wherein said pores are sized and shaped to allow said at least one organic material to create interactions with said tendon and/or said ligament;
wherein said fillable body comprises one or more mechanical characteristics similar to one or more mechanical characteristics of said tendon and/or said ligament that has been fixed into which said implantable device is implanted;
wherein said one or more mechanical characteristics are one or more of:
d. Elastic Modulus (Young's Modulus) from 500 to 1500 megapascals (MPa);
e. Strain from 5% to 20%
wherein at least one part of said fillable body is configured to be used as a soft anchor.

2. (canceled)

3. The implantable device according to claim 1, wherein said fillable body is made of one or more of Polylactic Acid (PLA), Polyglycolic Acid (PGA), Poly (lactic-co-glycolic acid) (PLGA), Polycaprolactone (PCL), Collagen, Collagen-based materials, hydrogels, Polyethylene Terephthalate (PET), Polyester; Polypropylene (PP), Expanded Polytetrafluoroethylene (ePTEE) and Ultra High Molecular Weight Polyethylene (UHMWPE).

4-6. (canceled)

7. The implantable device according to claim 1, wherein said body comprises a length of from 20 mm to 40 mm and a width of from 3 mm to 35 mm.

8. (canceled)

9. The implantable device according to claim 1, wherein said implantable device further comprises one or more additional means for attaching said implantable device to a tissue; and wherein said one or more additional means for attaching said implantable device to said tissue is one or more of sutures, organic glues and bone anchors.

10. The implantable device according to claim 1, wherein said body further comprises at least one coating.

11. The implantable device according to claim 1, wherein said plurality of organic materials are one or more of bone marrow, bone marrow stem cells, osteogenic cells, pre-osteoblasts, osteoblasts, mesenchymal stem cells, bone marrow mesenchymal stem cells, blood derivatives like Platelet-rich plasma (PRP) and/or plasma-derived medicinal products (PDMPs) and/or Platelet-rich fibrin (PRF) and pluripotent stem cells.

12. The implantable device according to claim 1, wherein said plurality of organic materials are one or more of autografts, allografts and synthetic grafts.

13. The implantable device according to claim 1, further comprising one or more bone anchors attached to said fillable body.

14. (canceled)

15. A method for augmenting and/or reinforcing a tendon and/or a ligament inside a body of patient that has been fixed, comprising:
a. filling an implantable device with at least one organic material; wherein said implantable device comprises a fillable body comprises walls having pores comprising a size of from 50 μm to 300 μm; wherein said pores are sized and shaped to allow said at least one organic material to migrate out of said fillable body; and wherein said pores are sized and shaped to allow said at least one organic material to create interactions with said tendon and/or said ligament; and
b. implanting said filled implantable device on said tendon and/or said ligament;
wherein said method further comprises, before said filling, identifying a type of implantable device required that comprises one or more mechanical characteristics similar to one or more mechanical characteristics of said tendon and/or said ligament;
wherein said one or more mechanical characteristics are one or more of:
d. Elastic Modulus (Young's Modulus) from 500 to 1500 megapascals (MPa);
e. Strain from 5% to 20%;
wherein said implanting comprises implanting a part of said fillable body as a soft anchor into a bone; and
anchoring another part of said fillable body with at least one additional anchoring means.

16. (canceled)

17. The method according to claim 15, wherein said implanting comprises suturing said implantable device to a tissue.

18. (canceled)

19. The method according to claim 15, wherein said implanting further comprises:
a. extending said implantable device over said tissue; and
b. implanting a second bone anchor attached to a second side of said implantable device into a second bone.

20. The method according to claim 19, wherein said bone and said second bone are a same bone.

21. The method according to claim 15, wherein said implanting comprises extending said implantable device over said tissue.

22. The method according to claim 15, wherein said anchoring another part of said fillable body comprises implanting a second part of said implantable device as a soft anchor into a second bone.

23. The method according to claim 22, wherein said bone and said second bone are a same bone.
